(19) United States
(12) Patent Application Publication
(10) Pub. No.: US 2026/0165792 A1
(43) Pub. Date: Jun. 18, 2026

(54) SURGICAL NAVIGATION SYSTEM USING VISION CAMERAS

(71) Applicant: FoxEyes Corporation, Plano, TX (US)

(72) Inventors: Hongwon YOON, Allen, TX (US); Seulki KIM, Uiwang-si (KR); Ting-Yun FANG, Taipei City (TW); Jieun SANG, Irvine, CA (US)

(73) Assignee: FoxEyes Corporation, Plano, TX (US)

(21) Appl. No.: 19/260,996

(22) Filed: Jul. 7, 2025

Related U.S. Application Data
(60) Provisional application No. 63/733,779, filed on Dec. 13, 2024.

(51) Int. Cl. A61B 34/20; A61B 34/30; A61B 34/32; A61B 90/00

(52) U.S. Cl. CPC A61B 34/20; A61B 34/32; A61B 90/39; A61B 2034/2057; A61B 2034/301; A61B 2090/3937; A61B 2090/3983

(57) ABSTRACT

A surgical navigation system may include a robot arm configured such that a surgical instrument may be mounted at one end thereof, a fixation pin configured to be fixed to a surgical target and include a visual marker, a camera configured to capture the visual marker and generate an image, and a controller configured to identify, in the image, a reference object corresponding to the visual marker and, on the basis of the reference object, control movement of the robot arm.

### CROSS-REFERENCE TO RELATED PATENT APPLICATION

This application claims the benefit of U.S. Provisional Application No. 63/733,779, filed on Dec. 13, 2024, the disclosure of which is incorporated herein in its entirety by reference.

### TECHNICAL FIELD

Embodiments relate to a surgical navigation system that employs a visual marker and to a method for controlling the same.

### BACKGROUND

Modern medical technology continues to advance rapidly. In particular, in the field of surgery, interest in and demand for technologies that aim at rapid patient recovery and improvement in surgical quality are constantly increasing. For accurate and safe surgery, it is very important to identify in real time the anatomical structure of the patient and the position of surgical instruments during surgery. Accordingly, various medical imaging devices such as an endoscopic camera, a mobile X-ray fluoroscopy apparatus (C-arm), and an ultrasonic imaging apparatus are used in the operating room as auxiliary apparatuses, and surgical planning and execution may be performed on the basis of image information acquired by such surgical auxiliary apparatuses.

However, much time and effort are required for the surgeon to analyze and utilize the various information obtained by the surgical auxiliary apparatuses. In addition, there is a need to secure economic viability so that the apparatuses can be used in more medical settings while maintaining the accuracy and stability of the surgical auxiliary apparatuses and at the same time lowering complexity and introduction cost.

### SUMMARY

Embodiments provide a surgical navigation system that employs a visual marker and a method for controlling the same, which solve the above-described problems.

Embodiments may be implemented in various forms, including a method, an apparatus (system), and/or a computer-readable storage medium, including a computer-readable storage medium having a computer program stored thereon.

In some embodiments, a surgical navigation system may include a robot arm configured such that a surgical instrument is mounted at one end thereof, a fixation pin configured to be fixed to a surgical target and may include a visual marker, a camera configured to capture the visual marker and generate an image, and a controller configured to identify, in the image, a reference object corresponding to the visual marker and, on the basis of the reference object, control movement of the robot arm.

In some embodiments, the controller may be further configured to acquire, on the basis of the reference object, a target position of the surgical target and control movement of the robot arm so that the surgical instrument reaches the target position.

In some embodiments, the image may include a plurality of images generated by capturing the visual marker from a plurality of viewpoints by the camera, and the controller is further configured to calculate position information of the visual marker on the basis of the plurality of images.

In some embodiments, the controller may be configured to acquire a first medical image including at least a portion of the surgical target captured by a first imaging device and, on the basis of the reference object, composite the surgical instrument onto the first medical image to generate a first composite image.

In some embodiments, the controller may be configured to acquire a second medical image including at least a portion of the surgical target captured by a second imaging device and, on the basis of the identified reference object, composite the surgical instrument onto the second medical image to generate a second composite image.

In some embodiments, the camera may include at least one of a 2D image sensor and a 3D image sensor.

In some embodiments, the camera may be mounted to the robot arm.

In some embodiments, a surgical navigation system may include a robot arm configured such that a surgical instrument is mounted at one end thereof, a fixation pin configured to be fixed to a surgical target and may include a visual marker, a camera configured to capture the visual marker and generate an image, and a controller configured to identify, in the image, a reference object corresponding to the visual marker and, on the basis of the reference object, determine whether the surgical target has moved.

In some embodiments, the image may include a first-time image generated by capturing the visual marker at a first point in time and a second-time image generated by capturing the visual marker at a second point in time later than the first point in time, and the controller is further configured to calculate first position information of the visual marker on the basis of the first-time image, calculate second position information of the visual marker on the basis of the second-time image, and determine whether the surgical target has moved on the basis of the first position information and the second position information.

In some embodiments, the controller may be further configured to control movement of the robot arm on the basis of the reference object.

In some embodiments, a surgical navigation system may include a robot arm configured such that a surgical instrument is mounted at one end thereof, a fixation pin configured to be fixed to a surgical target and may include a visual marker, a first camera configured to capture the visual marker from a first viewpoint and generate a first image, a second camera configured to capture the visual marker from a second viewpoint different from the first viewpoint and generate a second image, and a controller configured to identify, in the first image, a first reference object corresponding to the visual marker, identify, in the second image, a second reference object corresponding to the visual marker, control movement of the robot arm on the basis of the first reference object, and determine whether the surgical target has moved on the basis of the second reference object.

In some embodiments, the second image may include a (2-1)-th image generated by capturing the visual marker at a first point in time, and a (2-2)-th image generated by capturing the visual marker at a second point in time later than the first point in time, and wherein the controller is further configured to calculate first position information of the visual marker on the basis of the (2-1)-th image, calculate second position information of the visual marker on the basis of the (2-2)-th image, and determine whether the surgical target has moved on the basis of the first position information and the second position information.

In some embodiments, in response to determining that the surgical target has moved, the controller may be further configured to output an alarm related to adjustment of a position of the surgical target or of the robot arm.

In some embodiments, the controller may be further configured to calculate movement information of the visual marker on the basis of the first position information and the second position information and, in response to determining that the surgical target has moved, control movement of the robot arm on the basis of the movement information of the visual marker.

In some embodiments, a method for controlling a surgical navigation system, the method being executed by at least one processor, may include acquiring a medical image including at least a portion of a surgical target captured by a first imaging device, generating a first image by capturing, at a first viewpoint via a first camera, a visual marker included in a fixation pin configured to be fixed to the surgical target, generating a second image by capturing, at a second viewpoint different from the first viewpoint via a second camera, the visual marker, identifying, in the first image, a first reference object corresponding to the visual marker, identifying, in the second image, a second reference object corresponding to the visual marker, controlling movement of a robot arm configured such that a surgical instrument is mounted at one end thereof on the basis of the first reference object, and determining whether the surgical target has moved on the basis of the second reference object.

According to some embodiments, a composite image generated in real time may be provided to the surgeon through a display device or the like, allowing the surgeon to check the exact position of the surgical instrument in real time and thereby perform surgery accurately and safely.

According to some embodiments, the robotic arm may reach the target position more accurately, the alignment accuracy of the composite image may be further improved, and overall surgical quality may be enhanced.

According to some embodiments, because only a single visual marker is fixed to the surgical target, the surgical preparation process may be simplified and invasion of the surgical site may be minimized.

According to some embodiments, precise guidance of the robotic arm and position tracking of the surgical target are possible with only a single visual marker attached to a single fixation pin, so that pre-operative preparation time is reduced and working space for the surgeon or the robotic arm is maximized.

According to some embodiments, the surgical navigation system may reduce computation time and cost through position or coordinate transformation operations using a single visual marker, ensuring real-time operation of the system.

According to some embodiments, because sufficient accuracy can be provided even without an expensive multi-camera system, the introduction cost of the system may be reduced and maintenance convenience may be increased.

The effects of the present disclosure are not limited to the effects mentioned above. Other effects not mentioned will be clearly understood by one of ordinary skill in the art from the statements of the claims.

### BRIEF DESCRIPTION OF THE DRAWINGS

Embodiments will be described below with reference to the attached drawings, in which like reference numerals denote like elements but are not limited thereto.

FIG. 1 is an overview diagram illustrating a schematic configuration of a surgical navigation system according to an embodiment.

FIG. 2 is a block diagram illustrating an internal configuration of a computing device according to an embodiment.

FIG. 3 is a diagram illustrating an example of a fixation pin according to an embodiment.

FIG. 4 is a diagram illustrating a state in which the fixation pin according to an embodiment is fixed to a surgical target.

FIGS. 5 and 6 are diagrams for explaining a method of acquiring a transformation matrix according to an embodiment.

FIGS. 7 and 8 are diagrams for explaining a method of acquiring a transformation matrix according to an embodiment of the present disclosure.

FIGS. 9-12 are diagrams for explaining a method of determining whether a surgical target has moved according to an embodiment.

FIG. 13 is a diagram illustrating the fixation pin and a medical image according to an embodiment.

FIG. 14 is a diagram illustrating the fixation pin and a medical image according to an embodiment.

FIGS. 15 and 16 are diagrams for explaining a method of generating a composite image according to an embodiment.

FIG. 17 is a flowchart illustrating an example of a method for controlling the surgical navigation system according to an embodiment.

### DETAILED DESCRIPTION

Specific details for implementing embodiments will be described in detail below with reference to the accompanying drawings. However, when a detailed description of well-known functions or configurations could obscure the gist of the disclosure, such description is omitted.

In the attached drawings, identical or corresponding components are denoted by identical reference numerals. In the following description of embodiments, repetitive description of identical or corresponding components may be omitted. Even if a description of a component is omitted, it is not intended that such a component is not included in an embodiment.

Advantages, features, and methods for achieving them will become apparent by reference to the embodiments described below together with the drawings. The disclosure is not limited to the embodiments set forth below but may be embodied in various different forms, and the embodiments are merely provided so that the disclosure may be thoroughly disclosed and fully conveyed to those of ordinary skill in the art.

Terms used herein are briefly described, and the disclosed embodiments will be described in detail. Although the terms used herein are selected from general terms currently widely used, the meanings thereof may vary depending on the intention of a technician in the field, precedents, or the emergence of new technologies. In specific cases, the applicant may select arbitrary terms, and in such cases, the meanings of the terms will be described in detail in the portion describing the invention. Therefore, the terms used herein should be defined on the basis of the meanings and concepts consistent with the entire contents of the present specification rather than a simple term name.

Singular expressions as used herein include plural expressions unless the context clearly indicates otherwise. Likewise, plural expressions include singular expressions unless the context clearly indicates otherwise. Throughout the specification, when a portion is described as “including” a component, the description does not exclude the presence of other components unless otherwise specified.

The term “module” or “unit” as used in the specification denotes a software or hardware component and performs a specific role, but is not limited to software or hardware. The “module” or “unit” may reside in an addressable storage medium and may be configured to reproduce one or more processors. Accordingly, by way of example, the “module” or “unit” may include components such as software components, object-oriented software components, class components and task components, processes, functions, attributes, procedures, subroutines, segments of program code, drivers, firmware, micro-code, circuitry, data, databases, data structures, tables, arrays, or variables. The functions provided in the components and “modules” or “units” may be combined into a smaller number of components and “modules” or “units” or further separated into additional components and “modules” or “units.”

According to an embodiment, the “module” or “unit” may be implemented by a processor and a memory. The term “processor” should be broadly interpreted to include a general-purpose processor, a central processing unit (CPU), a microprocessor, a digital signal processor (DSP), a controller, a microcontroller, or a state machine. In some environments, the “processor” may refer to an application-specific integrated circuit (ASIC), a programmable logic device (PLD), or a field-programmable gate array (FPGA). The “processor” may also refer to a combination of processing devices such as a combination of a DSP and a microprocessor, a combination of multiple microprocessors, a combination of one or more microprocessors combined with a DSP core, or any other such configuration. The term “memory” should be broadly interpreted to include any electronic component capable of storing electronic information. The “memory” may refer to various types of processor-readable media such as random access memory (RAM), read-only memory (ROM), non-volatile RAM (NVRAM), programmable ROM (PROM), erasable PROM (EPROM), electrically erasable PROM (EEPROM), flash memory, magnetic or optical data storage devices, or registers. If the processor can read information from and/or write information to the memory, the memory is referred to as being in electronic communication with the processor. Memory integrated in the processor is in electronic communication with the processor.

The terms “first,” “second,” “A,” “B,” “(a),” “(b),” etc., used in the embodiments below are used only for the purpose of distinguishing one component from another component, and do not limit the nature, order, or sequence of the components.

When a component is described as being ‘connected,’ ‘coupled,’ or ‘joined’ to another component, the component may be directly ‘connected,’ ‘coupled,’ or ‘joined’ to the other component, or another component may be ‘connected,’ ‘coupled,’ or ‘joined’ between the components.

In the disclosure, the expression “each of a plurality of A” may refer to each of all components included in the plurality of A, or may refer to each of some components included in the plurality of A.

The terms “comprises” and/or “comprising” as used in the embodiments below do not exclude the presence or addition of one or more other components, steps, operations, or elements.

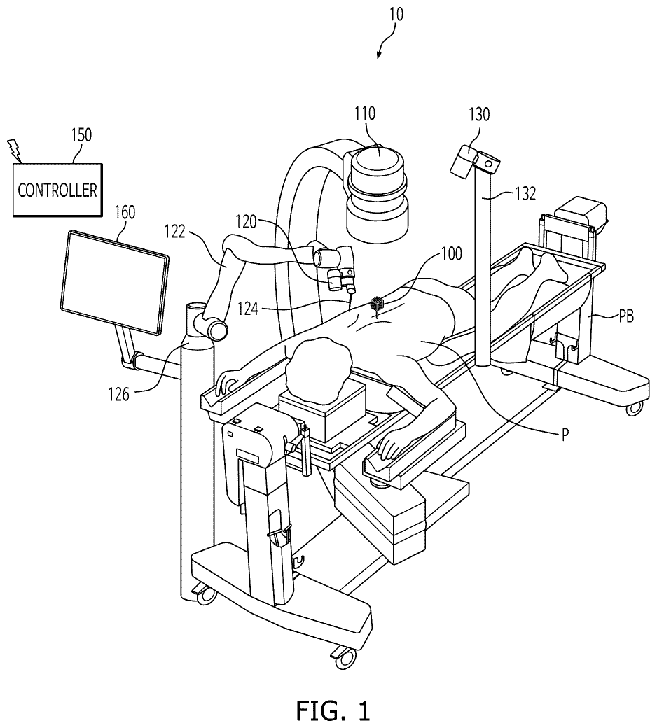

FIG. 1 is an overview diagram illustrating a schematic configuration of a surgical navigation system 10 according to an embodiment. The surgical navigation system 10 may include a fixation pin 100, a robotic arm 122, a first camera 120, and a controller 150. The surgical navigation system 10 may be used to assist a surgeon during surgery. For example, a patient may be placed on an operating table PB to undergo surgery. While performing surgery on the patient, the surgeon may be provided with information necessary for surgery from the surgical navigation system 10. In addition, the surgeon may control the robotic arm 122, to which a surgical instrument 124 is mounted, to perform the surgery.

The robotic arm 122 may have, for example, an articulated structure. By changing the posture of the articulated structure of the robotic arm 122, one end of the robotic arm 122 may move within a working space. A flange may be formed at one end of the robotic arm 122 for mounting a surgical instrument or an end effector. For example, an end effector may be mounted to the flange of the robotic arm 122. The surgical instrument 124 may be mounted to the end effector or directly to the flange. For example, the surgical instrument 124 may be a tool used for surgery on the patient and may include a cutting tool, a suturing tool, a drill, and the like.

In an embodiment, the robotic arm 122 may be mounted to a base 126. One end of the robotic arm 122 has the surgical instrument mounted thereto, and the other end of the robotic arm 122 may be connected to the base 126. The base 126 may be mounted to a mobility unit and may be movable. Alternatively, the base 126 may be fixed in position relative to the operating table PB.

The surgical navigation system 10 may include the fixation pin 100 configured to be fixed to a surgical target P. One end of the fixation pin 100 may be formed sharply so as to be easily inserted into the surgical target P. For example, the surgical target P may be a patient, and the fixation pin 100 may be fixed to a bony tissue (e.g., a vertebra) of the patient.

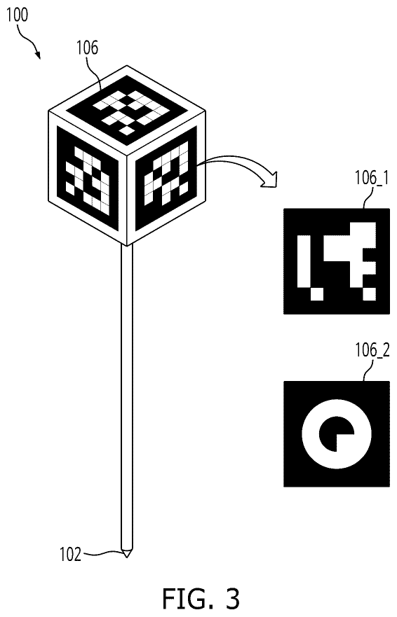

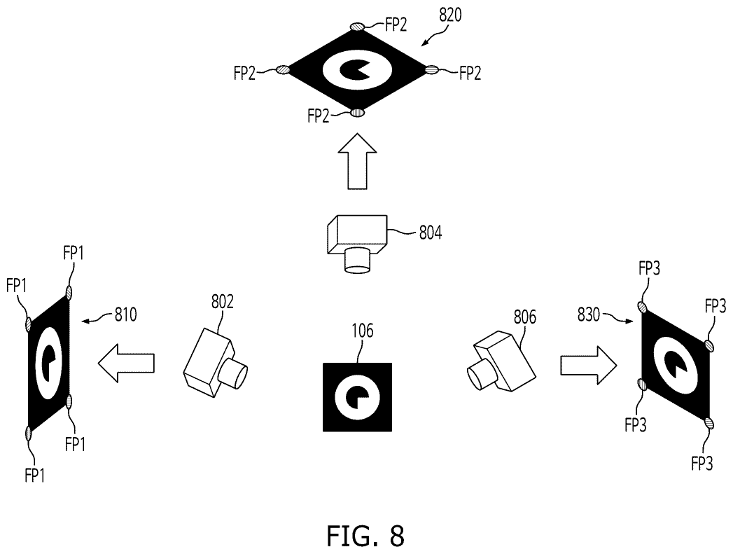

The fixation pin 100 may include a visual marker. For example, one end of the fixation pin 100 may be inserted into the surgical target P, and a visual marker may be attached to the other end of the fixation pin 100. The visual marker may be composed of a geometric pattern, a color combination, or a plurality of identifiable points. For example, the visual marker may include an ArUco marker, a QR code, or a barcode. Specific examples of the visual marker will be described in detail with reference to FIGS. 3 and 8.

The first camera 120 may be configured to capture the visual marker and generate a first image. For example, the first camera 120 may capture at least a portion of the surgical target P together with the visual marker. The first image generated by the first camera 120 may be transmitted to the controller 150.

In an embodiment, the first camera 120 may be mounted to the robotic arm 122. For example, the first camera 120 may be mounted to one end of the robotic arm 122 or to a region of the robotic arm 122 adjacent to the surgical instrument 124. The viewpoint of the first camera 120 may move or change in association with movement of the robotic arm 122. As the robotic arm 122 moves or its posture changes, the visual marker may enter or leave the field of view of the first camera 120.

In an embodiment, the surgical navigation system 10 may further include a second camera 130. The second camera 130 may be configured to capture the visual marker and generate a second image. For example, the second camera 130 may capture the surgical target P and/or the robotic arm 122 together with the visual marker. The second image generated by the second camera 130 may be transmitted to the controller 150.

In an embodiment, the viewpoint of the second camera 130 may differ from that of the first camera 120. For example, the first camera 120 is mounted to the robotic arm, and the field of view of the first camera 120 may be set according to the posture of the robotic arm. Meanwhile, the second camera 130 is installed with its position fixed relative to the operating table PB so as to capture the robotic arm 122 together with the visual marker. Referring to FIG. 1, the second camera 130 may be fixed by a camera support 132 of the operating table PB, but is not limited thereto. For example, the second camera 130 may be fixed to a mobile unit or the like and positioned adjacent to the operating table PB.

In an embodiment, the surgical navigation system 10 may further include a first imaging device 110 configured to capture the surgical target P to generate a first medical image. The first imaging device 110 may capture the surgical target P in real time while the surgeon performs surgery. The first medical image generated by the first imaging device 110 may be transmitted to the controller 150. The first medical image may also be output to a display device 160 and provided to the surgeon. In an example, the first imaging device 110 may be a 2D C-arm that irradiates X-rays, a 3D C-arm, a 2D O-arm, or a 3D O-arm. The first medical image may be an X-ray image.

In an embodiment, the surgical navigation system 10 may further include a second imaging device (not shown) configured to capture the surgical target P to generate a second medical image. The second imaging device may capture the surgical target P before the start of surgery. The controller 150 may acquire the second medical image generated in advance before the surgery starts. The second imaging device may capture the surgical target P to generate the second medical image. The second imaging device may be a computed tomography (CT) device or a magnetic resonance imaging (MRI) device.

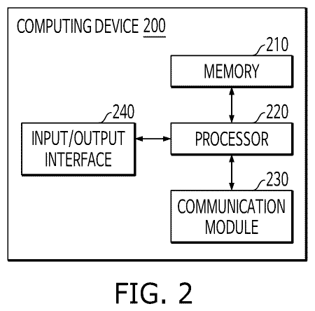

The controller 150 may be configured to overall manage or control the surgical navigation system 10. The controller 150 may control movement or posture change of the robotic arm 122. The controller 150 may receive the first image generated by the first camera 120 and/or the second image generated by the second camera 130. In an example, the controller 150 may be included in or may include a computing device (e.g., the computing device 200 of FIG. 2).

In an embodiment, the controller 150 may acquire and manage position information of components of the surgical navigation system 10. For example, the controller 150 may acquire relative position information of components of the surgical navigation system 10 with reference to the base 126 or the imaging devices (e.g., the first imaging device 110, the second imaging device). Below, a description is given in which the relative position information of the components of the surgical navigation system 10 is acquired with reference to the base 126 for convenience of explanation, but a method of acquiring relative position information of the components of the surgical navigation system 10 with reference to an imaging device may be substantially the same as or similar to the method of acquiring relative position information of the components of the surgical navigation system 10 with reference to the base 126. The controller 150 may calculate or acquire, in real time, position information of the robotic arm 122 (e.g., position information of the flange 128 of the robotic arm 122, position information of the surgical instrument 124, position information of the first camera 120, etc.) on the basis of a movement amount of the robotic arm 122 and/or joint angle information of the robotic arm 122. For example, the controller 150 may acquire initial position information of the robotic arm 122 on the basis of an initial position of the robotic arm 122 from a storage device installed in the system 10 or from an external device. The controller 150 may update the position information of the robotic arm 122 in response to movement of the robotic arm 122 while controlling movement of the robotic arm 122. In addition, the controller 150 may acquire position information of the second camera 130.

In an embodiment, the controller 150 may perform object recognition on an image (e.g., the first image generated by the first camera 120 and/or the second image generated by the second camera 130). For example, the controller 150 may identify, in the image, a reference object corresponding to the visual marker. A method by which the controller 150 identifies the reference object is described in detail with reference to FIG. 8.

In an embodiment, the first camera 120 and/or the second camera 130 may include at least one of a 2D image sensor and a 3D image sensor. The controller 150 may analyze an image generated by the 2D image sensor to calculate two-dimensional position information (e.g., X and Y coordinates) of a specific object included in the image. In this case, the controller 150 may calculate three-dimensional position information (e.g., X, Y, and Z coordinates) together with the two-dimensional position information of the specific object by using the identified reference object. Alternatively or additionally, the controller 150 may acquire three-dimensional position information of a specific object included in an image generated by the 3D image sensor.

In an embodiment, the position information may include posture information or direction information. For example, the direction information may include at least one of pitch, yaw, and roll. That is, the position information may include at least part of six-degree-of-freedom (6-DoF) information. For example, the position information may include X, Y, and Z coordinates and rotation-angle information.

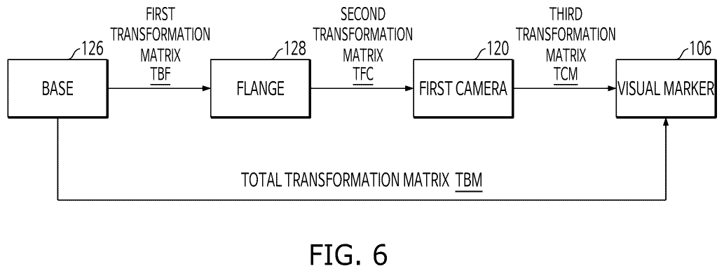

In an embodiment, the controller 150 may control movement of the robotic arm on the basis of the reference object. For example, the controller 150 may acquire or calculate a target position of the surgical target P on the basis of position information of the reference object. The target position may be predetermined with reference to a coordinate system based on the base 126 or an imaging device. For example, the target position may be position information calculated or determined on the basis of a shape of the visual marker included in a medical image. The controller 150 may control movement of the robotic arm 122 so that the surgical instrument 124 reaches the target position. A method of acquiring the target position is described in detail with reference to FIG. 6.

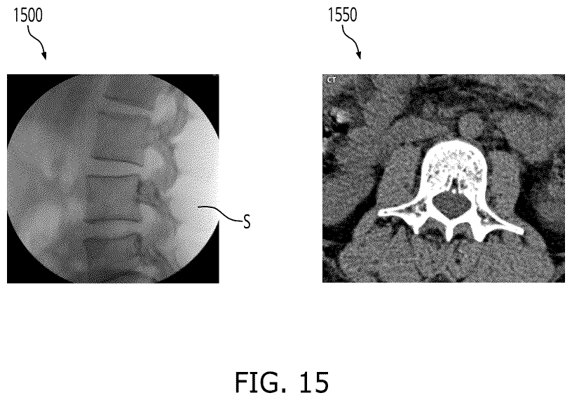

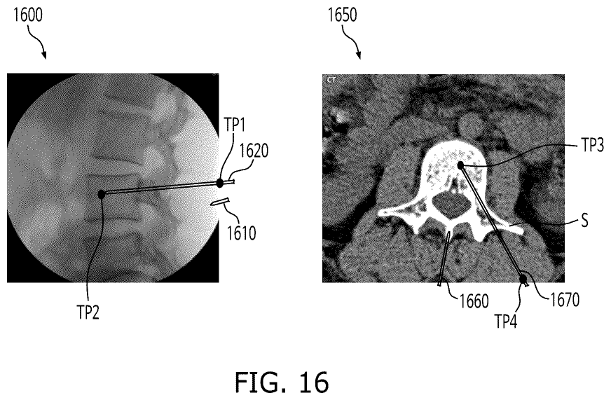

In an embodiment, the controller 150 may generate a first composite image by compositing the surgical instrument 124 onto the first medical image on the basis of the reference object. For example, the controller 150 may calculate a position of the surgical instrument 124 relative to the visual marker in the first medical image on the basis of position information of the reference object. The controller 150 may overlap a shape of the surgical instrument 124 with the first medical image on the basis of the calculated position of the surgical instrument 124 to generate the first composite image. As another example, the controller 150 may calculate a position of the surgical instrument 124 on the basis of position information of the reference object with reference to the base 126 or an imaging device. The position of the surgical instrument 124 calculated with reference to the base 126 or the imaging device may be expressed with reference to the first imaging device 110 (or a reference point of the first imaging device 110) on the basis of position information of the reference object. The controller 150 may superimpose a shape of the surgical instrument 124 onto the first medical image in real time on the basis of the position of the surgical instrument 124 expressed with reference to the first imaging device 110 (or the reference point of the first imaging device 110) to generate the first composite image. In addition, the controller 150 may calculate a position of the surgical instrument 124 on a first medical image captured during surgery on the basis of the reference object. The controller 150 may generate a second composite image related to the surgical site by superimposing a shape of the surgical instrument 124 onto a second medical image through alignment between the second medical image, which is acquired before the surgery, and the position of the surgical instrument 124 on the first medical image captured during the surgery. The second composite image may be updated in real time to provide visual information about a current position and orientation of the surgical instrument 124 to the surgeon. A method of generating the first composite image and the second composite image is described in detail with reference to FIGS. 15 and 16.

Specifically, the controller 150 may align coordinate systems of the imaging devices (e.g., the first imaging device 110 and the second imaging device), the base 126, and the cameras by using the reference object. Therefore, the controller 150 may generate a composite image (e.g., the first composite image or the second composite image) by calculating the current position of the surgical instrument 124 in a medical image. In addition, the controller 150 may calculate a target position for the surgical instrument 124 to reach.

FIG. 2 is a block diagram illustrating an internal configuration of a computing device 200 according to an embodiment of the present disclosure. The computing device 200 may include a memory 210, a processor 220, a communication module 230, and an input/output interface 1. The computing device 200 may be configured to communicate information and/or data over a network by using the communication module 230. For example, the computing device 200 may include the controller (e.g., the controller 150 of FIG. 1) or may be included in the controller (e.g., the controller 150 of FIG. 1).

In an embodiment, a user (e.g., a surgeon) may use the computing device 200 to receive surgical assistance. For example, the user may control a robotic arm (e.g., the robotic arm 122 of FIG. 1) by using the computing device 200. The user may obtain a composite image, in which a surgical instrument or the like is composited on an image of the surgical target, through a display device (e.g., the display device 160 of FIG. 1) by using the computing device 200.

The memory 210 may include any non-transitory computer-readable recording medium. In an embodiment, the memory 210 may include a permanent mass-storage device such as RAM, ROM, a disk drive, a solid-state drive (SSD), or a flash memory. As another example, a permanent mass-storage device such as ROM, SSD, flash memory, or a disk drive may be included in the computing device 200 as a separate permanent storage device distinct from the memory. The memory 210 may store an operating system and at least one program code (for example, code for controlling movement of the robotic arm, generating composite images, etc.) executed in the computing device 200.

Such software components may be loaded from a computer-readable recording medium separate from the memory 210. The separate computer-readable recording medium may include a recording medium that can be directly connected to the computing device 200, such as a floppy drive, a disk, a tape, a DVD/CD-ROM drive, or a memory card. As another example, the software components may be loaded into the memory 210 via the communication module 230, not via a computer-readable recording medium. For example, at least one program may be loaded into the memory 210 on the basis of installation files provided through the communication module 230 by a file distribution system that distributes file installation packages for developers or for distributing application installation files.

The processor 220 may be configured to process instructions of a computer program by performing basic arithmetic, logic, and input/output operations. Instructions may be provided via the memory 210 or the communication module 230 by a user terminal (not shown) or another external system. For example, the processor 220 may identify a reference object corresponding to the visual marker in an image. The processor 220 may control movement of the robotic arm on the basis of the reference object. The processor 220 may generate a composite image by compositing a target object onto a medical image on the basis of the reference object.

The communication module 230 may provide a configuration or function for the computing device 200 to communicate information and/or data with the cameras (e.g., the first camera 120 and the second camera 130 of FIG. 1), imaging devices (e.g., the first imaging device 110 of FIG. 1, etc.), a user terminal (not shown), or an external system over a network, and may provide a configuration or function for the computing device 200 to communicate with an external system (such as a separate cloud system). By way of example, control signals, instructions, data, etc., provided under control of the processor 220 of the computing device 200 may be transmitted to the user terminal and/or the external system through the communication module 230 and the network via the communication module of the user terminal and/or the external system.

The input/output interface 240 of the computing device 200 may be a means for interfacing between the computing device 200 and an input or output device (not shown) connected to, or included in, the computing device 200. In FIG. 2, the input/output interface 240 is illustrated as a component separate from the processor 220, but is not limited thereto and may be configured to be included in the processor 220. The computing device 200 may include more components than the components of FIG. 2. However, most conventional components need not be explicitly illustrated.

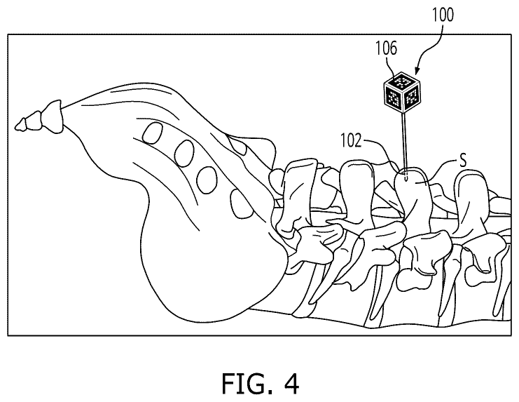

FIG. 3 is a diagram illustrating an example of the fixation pin 100 according to an embodiment. FIG. 4 is a diagram illustrating a state in which the fixation pin 100 according to an embodiment is fixed to a surgical target.

The fixation pin 100 may include a tip portion 102 and a visual marker 106. At least part of the fixation pin 100 (e.g., the tip portion 102) may be inserted into and fixed to the surgical target. At least another part of the fixation pin 100 (e.g., the visual marker 106) may protrude from the surgical target and visually indicate the location at which the fixation pin 100 is inserted into the surgical target.

Referring to FIG. 3, the fixation pin 100 may have a shape including a body portion extending in one direction and a head portion formed at one end of the body portion. The body portion is inserted into the surgical target, and the head portion, to which the visual marker 106 is attached, may be exposed outside the surgical target. However, the shape of the fixation pin 100 is not limited thereto, and the fixation pin 100 may have various shapes that are fixed to the surgical target and support or expose the visual marker 106.

In an embodiment, the tip portion 102 may be formed at one end of the fixation pin 100. The tip portion 102 may have a sharp shape so as to be inserted into bony tissue such as a vertebra S. For example, the tip portion 102 may have a threaded shape.

In an embodiment, the visual marker 106 may be located at an end of the fixation pin 100 opposite to the tip portion 102. When the fixation pin 100 is fixed with the tip portion 102 inserted into the surgical target, the visual marker 106 may protrude outside the surgical target. Referring to FIG. 3, the other end of the fixation pin 100 may be formed as a head portion that is flat in a direction perpendicular to the direction in which the body portion extends, and the visual marker 106 may be attached to the head portion. However, the disclosure is not limited to this, and the head portion may have a polyhedral shape, and one or more visual markers 106 may be attached to one or more faces of the polyhedron. That is, one fixation pin 100 may have one visual marker 106 or a plurality of visual markers 106 attached thereto.

In an embodiment, the visual marker 106 may have a predetermined visual pattern. For example, the visual marker 106 may be composed of a geometric pattern, a color combination, or a plurality of identifiable points. For example, the visual marker 106 may include an ArUco marker 106_1, a design marker 106_2, a QR code, or a barcode. In an image in which the visual marker 106 is captured, the visual marker 106 may be identified by the controller as a reference object.

Referring to FIG. 4, a distal portion 102 of the fixation pin 100 may be inserted into a spinous process S of the vertebra. At least a portion of the fixation pin 100 including the distal portion 102 may be inserted into the vertebra S. The visual marker 106 may be exposed outside the surgical target. Although only the vertebra S is illustrated in FIG. 4, the surgical target may include muscle, skin, and the like located around the vertebra S. Even in such a case, the visual marker 106 may be exposed outside the muscle or skin.

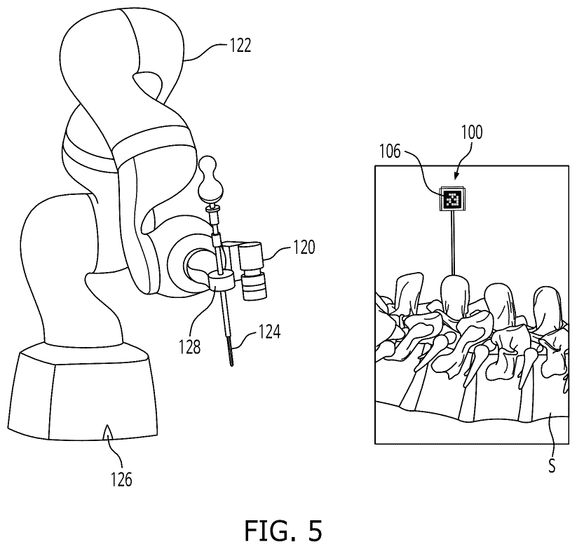

FIGS. 5 and 6 are diagrams illustrating a method of acquiring a transformation matrix according to an embodiment of the present disclosure. FIG. 5 may present an overall view of the robot arm 122 connected to the base 126 and of the fixation pin 100 fixed to the surgical target. FIG. 6 is a block diagram illustrating relationships among transformation matrices.

Referring to FIG. 5, a flange 128 may be formed at one end of the robot arm 122. The surgical instrument 124 may be mounted to the flange 128. The first camera 120 is positioned beside the flange 128 and may be mounted to the robot arm 122.

To express the position of a first object (e.g., the base 126, the flange 128, the first camera 120, the surgical instrument 124, or the visual marker 106), a second object (e.g., the base 126, the flange 128, the first camera 120, the surgical instrument 124, or the visual marker 106) may be used. For example, position information of the first object may be expressed in a relative relationship with respect to a reference point of the second object. In an example, the position information of the first object may be expressed in a coordinate system that uses the reference point of the second object as an origin. The reference point of the second object may be variously set at a portion or region of the second object. For example, the reference point of the second object may be set at a center of gravity, a centroid, a center of one face, or the most distal end of the second object. For convenience of explanation, when the coordinate system of the second object (or the reference point of the second object) is used to express the position information of the first object, the second object is described as a reference; however, unless otherwise specified, the second object may be understood to include its reference point. The coordinate system of the second object may denote a coordinate system that uses the reference point of the second object as an origin.

In an embodiment, the controller may acquire or determine position information of the flange 128 with reference to the base 126. For example, the controller may acquire initial position information of the flange 128 according to an initial position of the robot arm 122. As the controller controls movement of the robot arm 122, the controller may update the position information of the flange 128. Accordingly, the controller may acquire position information of the flange 128 in real time.

In an embodiment, the controller may acquire the predetermined position information of the first camera 120, etc., with reference to the flange 128. For example, the first camera 120 may be mounted at a predetermined position on the robot arm 122 with reference to the flange 128. The controller may acquire the predetermined position information of the first camera 120 with reference to the flange 128. In an example, the position information predetermined with reference to the flange 128 may be precisely obtained through hand-eye calibration.

In an embodiment, the controller may acquire parameters of the first camera 120. For example, a reference point (e.g., an optical center) of the first camera 120 may be set. The controller may acquire or determine focal length and position information of the optical center on the basis of the reference point of the first camera 120. In addition, the controller may acquire or determine distortion coefficients for correcting nonlinear distortion of an image. The controller may acquire parameters calculated on the basis of the focal length, the position information of the optical center, and/or the distortion coefficients. The parameters of the first camera 120 may be calculated in advance through camera calibration. On the basis of the parameters of the first camera 120, the controller may acquire or determine position information of an object included in a captured image with respect to the reference point of the first camera 120.

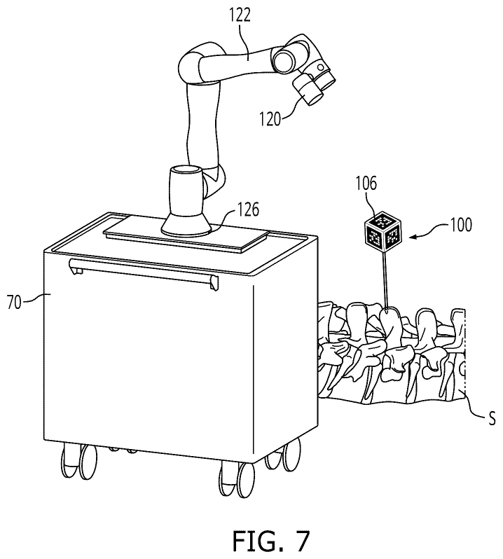

The fixation pin 100 to which the visual marker 106 is attached may be fixed to the surgical target. Referring to FIG. 5, the fixation pin 100 may be fixed to a vertebra S of a patient. In an embodiment, the controller may acquire a first image in which the visual marker 106 is captured by the first camera 120. The controller may perform object recognition on the first image and identify a reference object corresponding to the visual marker 106. On the basis of the parameters of the first camera 120, the controller may acquire or determine position information of the visual marker 106. The position information of the visual marker 106 may be determined or expressed with reference to the reference point of the first camera 120. A method of identifying the reference object in the first image and acquiring the position information of the visual marker will be described in detail with reference to FIGS. 7 and 8.

In an embodiment, the controller may acquire target position information on the basis of the visual marker 106. The target position information may be predetermined. For example, the target position information may be a target point to which the surgical instrument 124 is to move. The target position information may be expressed as a matrix such as TtM and may be predetermined on the basis of a medical image. TtM may be a matrix representing target position information expressed with reference to the visual marker 106. In superscripts and subscripts, M may denote the coordinate system of the visual marker and t may denote the target position.

In an embodiment, the controller may transform the target position information expressed with reference to the visual marker 106 into target position information expressed with reference to the base 126 by using transformation matrices. The controller may control the robot arm 122 on a coordinate system set with reference to the base 126. The controller may move the robot arm 122 to the target position by using the target position information expressed with reference to the base 126. A method of calculating the transformation matrices will be described in detail with reference to FIG. 6.

FIG. 6 illustrates coordinate-system transformation relationships among the base 126, the flange 128, the first camera 120, and the visual marker 106. Position information expressed with reference to the base 126 may be transformed into position information expressed with reference to the flange 128 by using a first transformation matrix TBF. Position information expressed with reference to the flange 128 may be transformed into position information expressed with reference to the first camera 120 by using a second transformation matrix TFC. Position information expressed with reference to the first camera 120 may be transformed into position information expressed with reference to the visual marker 106 by using a third transformation matrix TCM. Position information expressed with reference to the base 126 may be transformed into position information expressed with reference to the visual marker 106 by using a total transformation matrix TBM. The transformation matrix may be a 4×4 matrix used to transform position information expressed in one coordinate system into another coordinate system.

In an embodiment, the first transformation matrix TBF may be calculated on the basis of position information of the flange 128 expressed with reference to the base 126. In addition, the second transformation matrix TFC may be calculated on the basis of position information of a reference point of the first camera 120 expressed with reference to the flange 128. In an example, the second transformation matrix TFC may be determined by hand-eye calibration. For example, the second transformation matrix TFC may be predetermined on the basis of position information of the first camera 120 expressed with reference to the flange 128 and on the basis of parameters of the first camera 120. In addition, the third transformation matrix TCM may be calculated on the basis of position information of the visual marker 106 expressed with respect to the reference point of the first camera 120. The position information of the visual marker 106 expressed with respect to the reference point of the first camera 120 may be calculated by using the identified reference object and the parameters of the first camera 120. The position information of the visual marker 106 expressed with respect to the reference point of the first camera 120 may be calculated through a perspective-n-point (PnP) algorithm.

In an embodiment, the total transformation matrix TBM may be computed based on the first transformation matrix TBF, the second transformation matrix TFC, and the third transformation matrix TCM. For example, the total transformation matrix TBM may be computed as a product of the first transformation matrix TBF, the second transformation matrix TFC, and the third transformation matrix TCM. Specifically, the total transformation matrix TBM may be calculated as TMB=TBF·TFC·TCM. In the equation, B denotes the coordinate system of the base 126, F denotes the coordinate system of the flange 128, and CA denotes the coordinate system of the first camera 120.

A first target-position matrix, which is target position information expressed with reference to the visual marker 106, may be denoted as TtM. In an example, the first target-position matrix may be determined when the target position information is predetermined. A second target-position matrix, which is target position information expressed with reference to the base 126, may be denoted as TtB. The second target-position matrix may be obtained by converting the first target-position matrix by using the total transformation matrix TBM. In an example, the second target-position matrix may be calculated as TtB=TBM·TtM. Through the calculated TtB, the controller may move the robot arm to the target position.

In an embodiment, the controller may identify a target object (e.g., skin of the surgical target or the surgical instrument) included in the first image and may calculate or acquire position information of the target object. The position information of the target object may be expressed with reference to the first camera 120. Such position information of the target object may be transformed so as to be expressed with reference to the base 126 by using the first transformation matrix TBF and the second transformation matrix TFC.

In an embodiment, an imaging device (e.g., the first imaging device 110 and the second imaging device described with reference to FIG. 1) may capture the surgical target to which the fixation pin is fixed and may generate a medical image (e.g., a real-time X-ray image or a CT image). A target position may be determined on the medical image on the basis of the visual marker. A third target-position matrix, which is target position information expressed with reference to the visual marker 106 on the medical image, may be denoted as TtMCT. The third target-position matrix may be converted into the second target-position matrix. For example, the second target-position matrix may be converted from the third target-position matrix as TtB=TMCTB·TtMCT. Here, TMCTB may be calculated as TMCTB=TFB·TCAF·TCTCA·TMCTCT. TCTCA may be a transformation matrix for converting from the reference point of the first camera 120 to the reference point of the imaging device (e.g., an optical focus of the imaging device or a reference point of a calibration device used for calibrating the imaging device). TCTCA may be predetermined during calibration of the imaging device or may be calculated on the basis of parameters of the first camera 120 and the medical image. TMCTCT may be a transformation matrix for converting from the reference point of the imaging device to the visual marker identified in the medical image. TMCTCT may be predetermined during calibration of the imaging device or may be acquired by identifying the visual marker in the medical image. As described above, when a target position is determined on a medical image (e.g., a CT image or an X-ray image) on the basis of the visual marker, the target position information may be expressed with reference to the base 126 by using TMCTB. In superscripts and subscripts, CT denotes the coordinate system of the imaging device, and MCT denotes the coordinate system of the visual marker when captured by the imaging device.

In an embodiment, the second imaging device may capture the surgical target and may generate a second medical image. For example, the second medical image may be generated without the visual marker being captured together with the surgical target. A target position may be determined on the second medical image with reference to a reference point of the second imaging device (e.g., an optical focus of the second imaging device or a point located on an exterior surface of the second imaging device). A fourth target-position matrix, which is target position information expressed with reference to the reference point of the second imaging device on the second medical image, may be denoted as TtCT2. In superscripts and subscripts, CT2 denotes the coordinate system of the second imaging device. In the superscripts and subscripts of a matrix representing a transformation relationship with the coordinate system of the second imaging device, CT denotes the coordinate system of the first imaging device. The fourth target-position matrix may be converted into the second target-position matrix. For example, the second target-position matrix may be converted from the fourth target-position matrix as TtB=TCTB·TCT2CT·TtCT2. TCTB may be a matrix for converting from the reference point of the first imaging device (e.g., an optical focus of the first imaging device or a reference point of a calibration device used for calibrating the first imaging device) to the reference point of the base 126. TCTB may be calculated in advance on the basis of positional relationships between the base 126 and the first imaging device and on the basis of information related to the reference point of the first imaging device. TCT2CT may be a matrix for converting from the reference point of the first imaging device to the reference point of the second imaging device. For example, a target object (e.g., osseous tissue) may be identified on the first medical image, and position information of the target object may be acquired with reference to the reference point of the first imaging device. In addition, the target object may be identified on the second medical image, and position information of the target object may be acquired with reference to the reference point of the second imaging device. By matching the position information of the target object acquired with reference to the reference point of the first imaging device to the position information of the target object acquired with reference to the reference point of the second imaging device, TCT2CT may be calculated in advance.

In an embodiment, each transformation matrix (e.g., the first transformation matrix TBF, the second transformation matrix TFC, and the third transformation matrix TCM) or the total transformation matrix TBM may be acquired either before the start of surgery or during surgery. For example, before surgery starts, the first camera 120 may capture the visual marker 106 multiple times, and the controller may calculate the transformation matrix or the total transformation matrix on the basis of images including the captured visual marker 106. The target-position matrix may be acquired or updated either before surgery starts or during surgery. When the target-position matrix is updated, the controller may calculate a target position (e.g., TtB) with reference to the base 126 by using the transformation matrix or the total transformation matrix.

FIGS. 7 and 8 are diagrams illustrating a method of acquiring a transformation matrix according to an embodiment of the present disclosure.

FIG. 7 may illustrate that the first camera 120 mounted on the robot arm 122 captures the visual marker 106 a plurality of times. The visual marker 106 may be attached to the fixation pin 100 fixed to the surgical target.

In an embodiment, the first camera 120 mounted on the robot arm 122 may capture the visual marker 106 a plurality of times. By controlling the robot arm 122, the position or posture of the first camera 120 may be changed for each capture. The first camera 120 may capture the visual marker 106 from different viewpoints at each capture. As the first camera 120 performs multiple captures, a plurality of first images may be generated. The plurality of first images may be images captured such that at least the visual marker 106 and at least a portion of the surgical target are included together.

FIG. 8 may illustrate examples of the plurality of first images 810, 820, 830 generated by multiple captures of the visual marker 106. In an example, the first camera 802 at a first viewpoint may capture the visual marker 106 and may generate a (1-1)-th first image 810. The first camera 804 at a second viewpoint may capture the visual marker 106 and may generate a (1-2)-th first image 820. The first camera 802 at a third viewpoint may capture the visual marker 106 and may generate a (1-3)-th first image 830. The first camera 802, 804, 806 may capture the visual marker 106 less than three times or more than three times from various viewpoints. Hereinafter, description is made on the basis of three first images 810, 820, 830. In FIG. 8, only shapes of the visual marker 106 are illustrated in the first images 810, 820, 830; however, various objects (e.g., a surgical site of the surgical target, etc.) including the visual marker 106 may be displayed in the first images 810, 820, 830.

In an embodiment, to calculate the transformation matrix (e.g., the third transformation matrix TCM described with reference to FIG. 6) or the total transformation matrix more accurately, the controller may acquire the plurality of first images 810, 820, 830. The controller may identify the visual marker 106 as a reference object in each of the plurality of first images 810, 820, 830. For example, the controller may identify a first reference object in the (1-1)-th first image 810, may identify a second reference object in the (1-2)-th first image 820, and may identify a third reference object in the (1-3)-th first image 830. The controller may acquire position information of feature points FP1, FP2, FP3 of each reference object. Referring to FIG. 8, the controller may identify a corner of the first reference object in the (1-1)-th first image 810 as the feature point FP1 for the first reference object. The controller may identify a corner of the second reference object in the (1-2)-th first image 820 as the feature point FP2 for the second reference object. The controller may identify a corner of the third reference object in the (1-3)-th first image 830 as the feature point FP3 for the third reference object. In FIG. 8, four feature points are identified for each reference object; however, fewer than four or more than four feature points may be identified for each reference object. The controller may acquire position information (e.g., X, Y, Z coordinates and rotation-angle information) of the plurality of feature points FP1, FP2, FP3.

In an embodiment, the controller may collectively use the parameters of the first camera and the position information of the plurality of feature points FP1, FP2, FP3 to calculate position information (e.g., X, Y, Z coordinates and rotation-angle information) of the visual marker. That is, position information (e.g., three-dimensional information and direction information) of the visual marker 106 may be calculated by performing triangulation on the basis of the position information of the respective feature points FP1, FP2, FP3. On the basis of the position information of the visual marker 106, the controller may calculate either a transformation matrix for converting from the first camera 120 to the visual marker 106 or the total transformation matrix. That is, by accurately calculating the transformation matrix or the total transformation matrix through the images generated by capturing the visual marker 106 multiple times, the controller may more precisely determine a target position expressed with reference to the base.

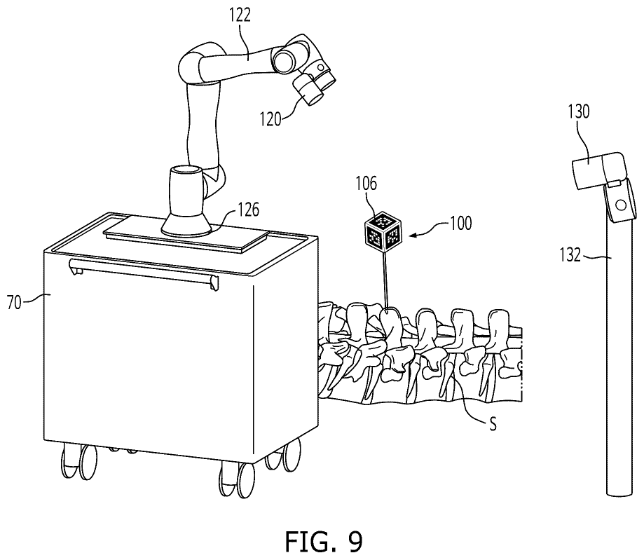

FIGS. 9 to 12 are diagrams illustrating a method of determining whether the surgical target P has moved according to an embodiment of the present disclosure.

FIG. 9 may illustrate that the first camera 120 and a second camera 130 capture the visual marker 106. The first camera 120 mounted on the robot arm 122 may capture the visual marker 106. The visual marker 106 may be attached to the fixation pin 100 fixed to the surgical target.

In an embodiment, the second camera 130 may be supported by a camera support 132 and may be disposed in a region adjacent to the surgical target. The second camera 130 may capture the surgical target, the first camera 120, and/or the robot arm 122 together with the visual marker 106. Referring to FIG. 9, the second camera 130 is illustrated as being fixed; however, the second camera 130 may be mounted to a mobile unit or the like and may be movable.

In an embodiment, during surgery, the second camera 130 may be disposed such that its viewpoint is directed toward the visual marker 106. In this case, during surgery, the visual marker 106 may be removed from the field of view of the first camera 120 as the robot arm 122 operates. When the visual marker 106 is not included in the field of view of the first camera 120, the first camera 120 may cease capturing; however, the present disclosure is not limited thereto. For example, the first camera 120 may operate continuously during surgery.

In FIG. 9, the surgical navigation system is illustrated as including the first camera 120 and the second camera 130; however, the present disclosure is not limited thereto. For example, the surgical navigation system may include only the second camera 130. In this case, the second camera 130 may perform functions or operations performed by the first camera 120. For example, instead of expressing locations of the surgical instrument, the visual marker 106, target positions, and imaging devices on a coordinate system with reference to the base 126 of the robot arm 122, a coordinate system may be set with reference to the camera support 132 of the second camera 130, and the coordinate system of the base 126 may be aligned thereto to express positions. In addition, instead of a transformation matrix and a total transformation matrix associated with the first camera 120, a transformation matrix and a total transformation matrix associated with the second camera 130 may be calculated. Through this, the target position may be expressed with reference to the coordinate system of the base 126. Then, the controller may move the surgical instrument to the target position calculated through the second camera 130. In addition, the controller may generate a composite image by compositing the surgical instrument onto the medical image. That is, the coordinate-system alignment method described with reference to FIGS. 5 and 6 may likewise be applied to the second camera 130 instead of the first camera 120.

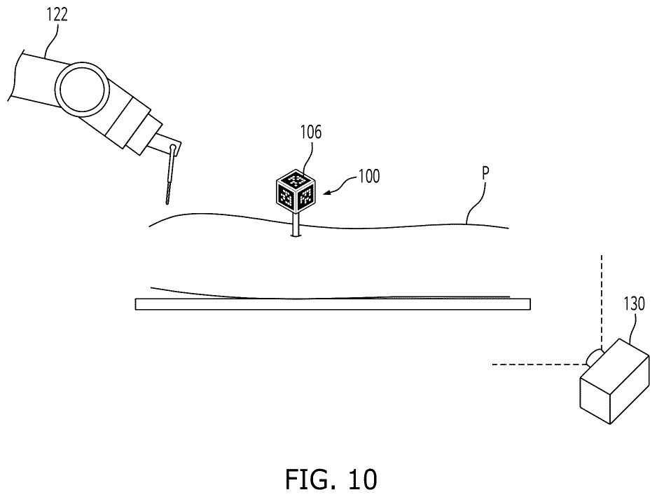

FIG. 10 illustrates that the second camera 130 captures the surgical target P at a first point in time. For example, the first point in time may be after the start of surgery. The second camera 130 may capture the visual marker 106 at the first point in time and may generate a first-time second image (2-1). On the basis of the (2-1) image, the controller may acquire first position information of the visual marker at the first point in time. The first position information of the visual marker may be calculated with reference to the base connected to the robot arm 122. A method of calculating the first position information of the visual marker may be understood in detail with reference to FIGS. 5 and 6.

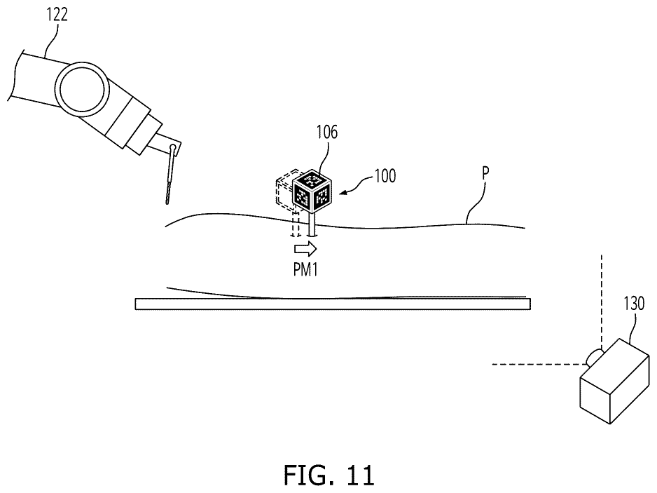

FIG. 11 illustrates that the second camera 130 captures the surgical target P at a second point in time after the first point in time. The second camera 130 may capture the visual marker 106 at the second point in time and may generate a second-time second image (2-2). On the basis of the (2-2) image, the controller may acquire second position information of the visual marker at the second point in time. The second position information of the visual marker may be calculated with reference to the base connected to the robot arm 122. A method of calculating the second position information of the visual marker may be understood in detail with reference to FIGS. 5 and 6.

In an embodiment, as time elapses from the first point in time to the second point in time, the fixation pin 100 may move. For example, the fixation pin 100 may move as the surgical target P to which the fixation pin 100 is fixed moves. The controller may determine whether the surgical target P has moved on the basis of the first position information and the second position information.

In an embodiment, when the controller determines that the surgical target P has moved, the controller may output an alarm related to adjustment of the position of the surgical target P or the robot arm 122. For example, the controller may output information related to adjustment of the position of the surgical target P or the robot arm 122 through a display device. As another example, the controller may output information related to adjustment of the position of the surgical target P or the robot arm 122 through an audio device connected to the controller. As yet another example, in response to determining that the surgical target P has moved, the controller may stop movement of the robot arm 122. The controller may control the robot arm 122 to move again in response to receiving a user input through a display device or an input device.

In an embodiment, the controller may calculate movement information PM1 of the visual marker on the basis of the first position information and the second position information. For example, the controller may calculate the movement information PM1 of the visual marker on the basis of a difference between the first position information and the second position information. In response to determining that the surgical target P has moved, the controller may output the movement information PM1 of the visual marker through a display device.

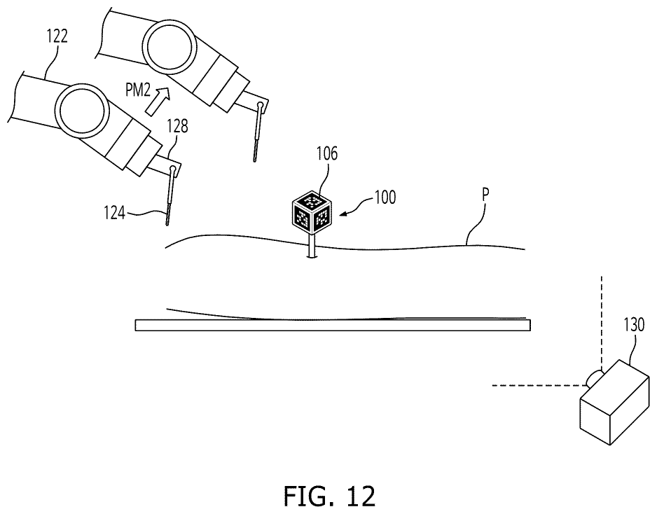

FIG. 12 illustrates that the second camera 130 captures the surgical target P at a third point in time after the second point in time. In an embodiment, in response to determining that the surgical target P has moved, the controller may control movement of the robot arm 122 on the basis of the movement information PM1 of the visual marker. For example, the controller may calculate movement information PM2 of the robot arm to compensate by the same displacement corresponding to the movement information PM1 of the visual marker. The controller may move the robot arm 122 on the basis of the movement information PM2 of the robot arm. Accordingly, a relative positional relationship between the surgical instrument 124 or the flange 128 of the moved robot arm 122 and the visual marker 106 may be substantially identical to a relative positional relationship between the surgical instrument 124 or the flange 128 and the visual marker 106 at the first point in time.

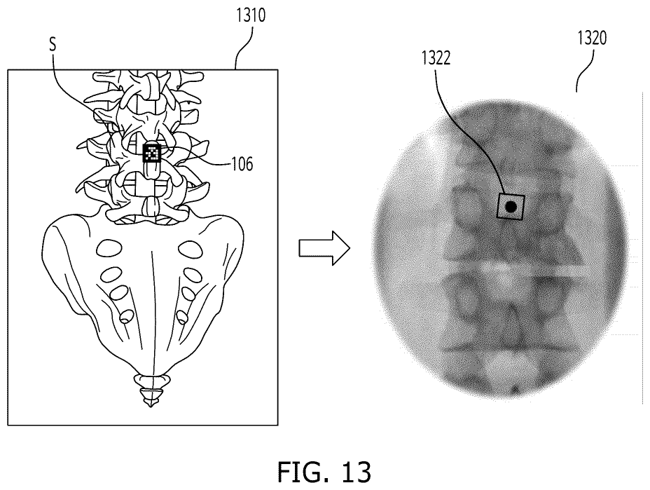

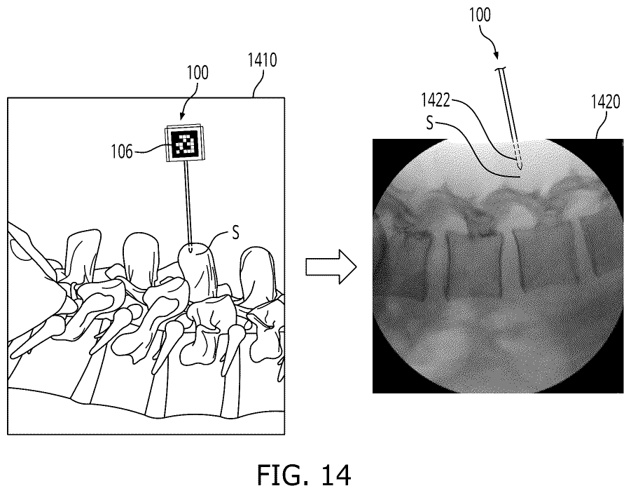

FIG. 13 is a diagram illustrating the fixation pin 100 and a medical image 1320 according to an embodiment of the present disclosure. FIG. 14 is a diagram illustrating the fixation pin 100 and a medical image 1420 according to an embodiment of the present disclosure.

Referring to FIG. 13, a first example 1310 may illustrate that the fixation pin 100 is inserted into the spinous process of the vertebra S when the visual marker 106 is viewed from the front. In an embodiment, the imaging device may capture the state in which the fixation pin 100 is inserted and may generate the medical image 1320. For example, the medical image 1320 may be an X-ray image. The medical image 1320 may include a first projection shape 1322 formed by capturing the visual marker 106.

In FIG. 13, the first projection shape 1322 is illustrated on the medical image 1320 to represent the visual marker 106; however, the present disclosure is not limited to the depiction in FIG. 13. For example, the first projection shape 1322 may be composited or the shape of the visual marker 106 may be further highlighted on the medical image 1320 to emphasize the visual marker 106. As another example, the first projection shape 1322 may not be formed on the medical image 1320, and a shape associated with the visual marker may be composited on a composite image.

In an embodiment, the controller may identify position information of the visual marker 106 on the medical image 1320. For example, the controller may recognize the first projection shape 1322 on the medical image 1320 and may acquire position information of the visual marker 106 with reference to the imaging device. The acquired position information of the visual marker 106 may be expressed with reference to the base by using the transformation matrices described with reference to FIGS. 5 and 6.

FIG. 14 illustrates a second example 1410 in which the fixation pin 100 is inserted into the spinous process of the vertebra S when the visual marker 106 is viewed from the side.

In an embodiment, the imaging device may capture the state in which the fixation pin 100 is inserted and may generate the medical image 1420. For example, the medical image 1420 may be an X-ray image. The medical image 1420 may include a second projection shape 1422 formed by capturing the fixation pin 100. The second projection shape 1422 may correspond to at least a portion of the fixation pin 100.

In FIG. 14, the second projection shape 1422 is illustrated on the medical image 1420 to represent the visual marker 106; however, the present disclosure is not limited to the depiction in FIG. 14. For example, the second projection shape 1422 may be composited or the shape of the fixation pin 100 may be further highlighted on the medical image 1420 to emphasize the fixation pin 100. As another example, the second projection shape 1422 may not be formed on the medical image 1420, and a shape associated with the fixation pin 100 may be composited on a composite image.

In an embodiment, the controller may acquire position information of the fixation pin 100. For example, as described with reference to FIG. 13, the controller may acquire position information of the visual marker 106. The controller may acquire physical-structure information (e.g., length, size, and width) of the fixation pin 100 in advance. On the basis of the position information of the visual marker 106 and the physical-structure information of the fixation pin 100, the controller may calculate position information of the fixation pin 100, and the position information of the fixation pin 100 may be expressed with reference to the base.

FIGS. 15 and 16 are diagrams illustrating a method of generating composite images 1600, 1650 according to an embodiment of the present disclosure. FIG. 15 may show medical images 1500, 1550 on which generation of the composite images 1600, 1650 is based. FIG. 16 may illustrate examples of the composite images 1600, 1650.

Referring to FIG. 15, a first medical image 1500 may be an image captured by a first imaging device (e.g., the first imaging device 110 of FIG. 1), and a second medical image 1550 may be an image captured by a second imaging device (e.g., a CT device). The first medical image 1500 may be an image capturing a lateral side of the vertebra S, and the second medical image 1550 may be an image capturing a cross-section of the vertebra S. The medical images 1500, 1550 may be captured before the start of surgery. For example, the medical images 1500, 1550 may be generated by capturing the surgical target before insertion of the fixation pin; however, the present disclosure is not limited thereto, and the medical images may be generated by capturing the surgical target after insertion of the fixation pin.

Referring to FIG. 16, a first composite image 1600 may be an image composited on the first medical image 1500, and a second composite image 1650 may be an image composited on the second medical image 1550.

In an embodiment, the controller may acquire position information of the visual marker. The controller may acquire physical-structure information of the fixation pin. On the basis of the position information of the visual marker and the physical-structure information of the fixation pin, the controller may calculate position information of the fixation pin. On the basis of the position information of the fixation pin and the physical-structure information of the fixation pin, the controller may composite first composite shapes 1610, 1660 corresponding to the shape of the fixation pin on the medical images 1500, 1550. Accordingly, the composite images 1600, 1650 may include the first composite shapes 1610, 1660 corresponding to the shape of the fixation pin. The first composite shapes 1610, 1660 may correspond to at least a portion of the fixation pin inserted into the vertebra S.

In an embodiment, the controller may acquire position information of the visual marker. The controller may acquire target position information on the basis of the position information of the visual marker. A method of acquiring the target position information may be understood on the basis of the description with reference to FIGS. 5 and 6. For example, the target position information may include position information of at least one target point TP1-TP4 to which the surgical instrument is to reach. The controller may control movement of the robot arm connected to the surgical instrument so that the surgical instrument reaches the target point. Accordingly, the surgical instrument may reach the target point.

In an embodiment, the controller may composite the surgical instrument on the medical images 1500, 1550. For example, the controller may acquire position information of the surgical instrument and position information of the visual marker. A method of acquiring the position information of the surgical instrument and the position information of the visual marker may be understood on the basis of the description with reference to FIGS. 5 and 6. The controller may acquire physical-structure information of the surgical instrument in advance. On the basis of the position information of the surgical instrument and the physical-structure information of the surgical instrument, the controller may composite second composite shapes 1620, 1670 corresponding to the shape of the surgical instrument on the medical images 1500, 1550.

Referring to FIG. 16, it may be confirmed on the composite images 1600, 1650 that the surgical instrument reaches at least one target point TP1-TP4. However, at least one target point TP1-TP4 may not be composited on the composite images 1600, 1650.

As described above, the controller may generate the composite images 1600, 1650 in real time. The composite images 1600, 1650 generated in real time may be provided to the surgeon through a display device or the like. A surgeon provided with the composite images 1600, 1650 may verify an exact position of the surgical instrument in real time and may perform surgery accurately and safely.

The robot arm may reach the target position more accurately, and alignment accuracy of the composite images 1600, 1650 may be further improved, so that overall surgical quality may be enhanced.

By fixing only a single visual marker to the surgical target, the surgical preparation process may be simplified and invasion of the surgical site may be minimized. In addition, precise guidance of the robot arm and position tracking of the surgical target are possible with only a single visual marker attached to a single fixation pin, so that pre-operative preparation time is reduced and working space for the surgeon or the robot arm is maximized.

Further, a system using multiple cameras or a plurality of visual markers requires complex calculations to generate composite images and to control movement of the robot arm, and considerable time may be consumed for the calculations. In contrast, the surgical navigation system according to the present disclosure may reduce computation cost through transformation operations using a single visual marker, and the system may operate in real time because less computation time is required.

The surgical navigation system according to an embodiment may be operated with only the first camera or the second camera. Therefore, sufficient accuracy can be provided even without an expensive multi-camera system, so that introduction cost of the system may be reduced and maintenance convenience may be increased.

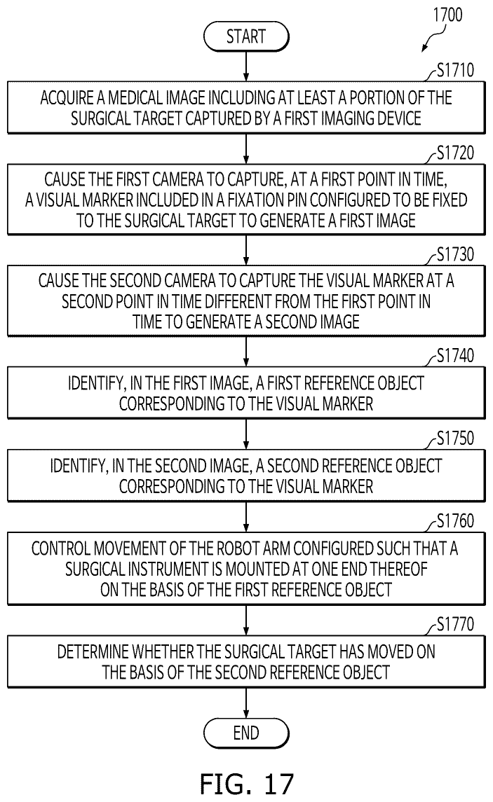

FIG. 17 is a flowchart illustrating an example of a method 1700 of controlling the surgical navigation system according to an embodiment of the present disclosure. The method 1700 of controlling the surgical navigation system may be performed by at least one processor included in the controller (e.g., the controller 150 of FIG. 1).

The method 1700 of controlling the surgical navigation system may start with acquiring a medical image including at least a portion of the surgical target captured by a first imaging device S1710.

In an embodiment, the controller may cause the first camera to capture, at a first point in time, a visual marker included in a fixation pin configured to be fixed to the surgical target to generate a first image S1720. The first camera may be mounted to the robot arm.

In an embodiment, the controller may cause the second camera to capture the visual marker at a second point in time different from the first point in time to generate a second image S1730. For example, the first camera and/or the second camera may include at least one of a 2D image sensor and a 3D image sensor.

In an embodiment, the controller may identify, in the first image, a first reference object corresponding to the visual marker S1740. For example, the image may include a plurality of images generated by capturing from a plurality of viewpoints by the camera. The controller may calculate position information of the visual marker on the basis of the plurality of images.

In an embodiment, the controller may identify, in the second image, a second reference object corresponding to the visual marker S1750.

In an embodiment, the controller may control movement of the robot arm configured such that a surgical instrument is mounted at one end thereof on the basis of the first reference object S1760. For example, the controller may acquire a target position of the surgical target on the basis of the first reference object and may control movement of the robot arm so that the surgical instrument reaches the target position.

In an embodiment, the controller may determine whether the surgical target has moved on the basis of the second reference object S1770. For example, the second image may include a (2-1) image generated by capturing the visual marker at the first point in time and a (2-2) image generated by capturing the visual marker at the second point in time after the first point in time. The controller may calculate first position information of the visual marker on the basis of the (2-1) image. The controller may calculate second position information of the visual marker on the basis of the (2-2) image. The controller may determine whether the surgical target has moved on the basis of the first position information and the second position information.

In an embodiment, in response to determining that the surgical target has moved, the controller may output an alarm related to adjustment of the position of the surgical target or the robot arm.

In an embodiment, the controller may calculate movement information of the visual marker on the basis of the first position information and the second position information. In response to determining that the surgical target has moved, the controller may control movement of the robot arm on the basis of the movement information of the visual marker.

In an embodiment, on the basis of at least one of the first reference object and the second reference object, the controller may composite the surgical instrument onto the first medical image to generate a first composite image.

In an embodiment, the controller may acquire a second medical image including at least a portion of the surgical target captured by a second imaging device. On the basis of at least one of the identified first reference object and the second reference object, the controller may composite the surgical instrument onto the second medical image to generate a second composite image.

The above-described methods may be provided as a computer-readable recording medium storing a computer program for execution on a computer. The medium may permanently store a program executable by a computer, or may temporarily store the program for execution or download. The medium may be a recording or storage means having various forms in which one or more pieces of hardware are combined, and is not limited to a medium directly connected to a computer system but may exist in a distributed manner over a network. Examples of the medium may include magnetic media such as hard disks, floppy disks, and magnetic tapes; optical-recording media such as CD-ROM and DVD; magneto-optical media such as floptical disks; and ROM, RAM, and flash memory configured to store program instructions. Other examples of the medium may include recording or storage media managed by app stores distributing applications or by various websites or servers supplying or distributing various software.

The methods, operations, and techniques of the present disclosure may be implemented by various means. For example, the techniques may be implemented in hardware, firmware, software, or a combination thereof. A person having ordinary skill in the art will understand that the various illustrative logical blocks, modules, circuits, and algorithm steps described in connection with the present disclosure may be implemented in electronic hardware, computer software, or combinations of both. To clarify interchangeability between hardware and software, the various illustrative components, blocks, modules, circuits, and steps have been described above primarily from a functional standpoint. Whether the functionality is implemented in hardware or in software depends on design requirements imposed on the specific application and overall system. A person having ordinary skill in the art may implement the described functionality in various ways for each specific application, and such implementations should not be construed as departing from the scope of the present disclosure.

In a hardware implementation, the processing units used to perform the techniques may be implemented in one or more application-specific integrated circuits (ASICs), digital signal processors (DSPs), digital signal processing devices (DSPDs), programmable logic devices (PLDs), field-programmable gate arrays (FPGAs), processors, controllers, microcontrollers, state machines, or combinations thereof.

Accordingly, the various illustrative logical blocks, modules, and circuits described in connection with the present disclosure may be implemented or executed by a general-purpose processor, a DSP, an ASIC, an FPGA, another programmable logic device, discrete gate or transistor logic, discrete hardware components, or any combination designed to perform the functions described herein. A general-purpose processor may be a microprocessor; alternatively, the processor may be any conventional processor, controller, microcontroller, or state machine. The processor may also be implemented as a combination of computing devices, for example a combination of a DSP and a microprocessor, multiple microprocessors, one or more microprocessors in conjunction with a DSP core, or any other such configuration.

In a firmware or software implementation, the techniques may be implemented as computer-executable instructions that are stored on a computer-readable medium and executed by one or more processors. The computer-readable medium may include any electronic component capable of storing electronic information, such as random-access memory (RAM), read-only memory (ROM), non-volatile RAM (NVRAM), programmable ROM (PROM), erasable PROM (EPROM), electrically erasable PROM (EEPROM), flash memory, magnetic or optical data storage devices, registers, or combinations thereof. When a processor can read information from and/or write information to the memory, the memory is said to be in electronic communication with the processor. A memory integrated into a processor is in electronic communication with the processor.

Software-implemented instructions or code may also be stored on or transmitted over any suitable computer-readable medium. Computer-readable media include both storage media and communication media that facilitate transfer of a computer program from one place to another. Storage media may be any available media that can be accessed by a computer, including RAM, ROM, EEPROM, CD-ROM or other optical-disk storage, magnetic-disk storage, or other magnetic-storage devices, or any other medium that can be used to carry or store desired program code in the form of instructions or data structures and that can be accessed by a computer.

When software is transmitted from a website, a server, or another remote source using coaxial cable, fiber-optic cable, twisted-pair, digital subscriber line (DSL), or wireless technologies such as infrared, radio, and microwave, the coaxial cable, fiber-optic cable, twisted-pair, DSL, or wireless technologies are included within the definition of a communication medium. Disks and discs, as used herein, include compact discs (CDs), laser discs, optical discs, digital versatile discs (DVDs), floppy disks, and Blu-ray discs, where disks generally reproduce data magnetically while discs reproduce data optically with lasers.

A software module may reside in RAM, flash memory, ROM, EPROM, EEPROM, registers, a hard disk, a removable disk, a CD-ROM, or any other known form of storage medium. The storage medium may be coupled to the processor so that the processor can read information from, and write information to, the storage medium. Alternatively, the storage medium may be integral to the processor. The processor and the storage medium may reside within an ASIC. The ASIC may reside within a user terminal. Alternatively, the processor and the storage medium may reside as discrete components within a user terminal.

Although the above-described embodiments have been explained as utilizing aspects of the present disclosure in one or more stand-alone computer systems, the present disclosure is not limited thereto. The embodiments may likewise be implemented in any computing environment, such as a networked or distributed computing environment. Furthermore, the aspects of the present disclosure may be implemented across multiple processing chips or devices, and storage may similarly be affected across multiple devices. Such devices may include personal computers, network servers, and portable devices.

While the present disclosure has been described in connection with certain embodiments, various modifications and changes may be made without departing from the scope of the present disclosure as will be understood by those having ordinary skill in the art. Such modifications and changes should be considered as falling within the scope of the claims appended hereto.

What is claimed is:

1. A surgical navigation system comprising:
a robot arm configured such that a surgical instrument is mounted at one end thereof;
a fixation pin configured to be fixed to a surgical target and comprising a visual marker;
a camera configured to capture the visual marker and generate an image; and
a controller configured to identify, in the image, a reference object corresponding to the visual marker and, on the basis of the reference object, control movement of the robot arm.

2. The surgical navigation system as claimed in claim 1, wherein the controller is further configured to acquire, on the basis of the reference object, a target position of the surgical target and control movement of the robot arm so that the surgical instrument reaches the target position.

3. The surgical navigation system as claimed in claim 1, wherein the image comprises a plurality of images generated by capturing the visual marker from a plurality of viewpoints by the camera, and the controller is further configured to calculate position information of the visual marker on the basis of the plurality of images.

4. The surgical navigation system as claimed in claim 1, wherein the controller is configured to acquire a first medical image including at least a portion of the surgical target captured by a first imaging device and, on the basis of the reference object, composite the surgical instrument onto the first medical image to generate a first composite image.

5. The surgical navigation system as claimed in claim 4, wherein the controller is configured to acquire a second medical image including at least a portion of the surgical target captured by a second imaging device and, on the basis of the identified reference object, composite the surgical instrument onto the second medical image to generate a second composite image.

6. The surgical navigation system as claimed in claim 1, wherein the camera comprises at least one of a 2D image sensor and a 3D image sensor.

7. The surgical navigation system as claimed in claim 1, wherein the camera is mounted to the robot arm.

8. A surgical navigation system comprising:
a robot arm configured such that a surgical instrument is mounted at one end thereof;
a fixation pin configured to be fixed to a surgical target and comprising a visual marker;
a camera configured to capture the visual marker and generate an image; and
a controller configured to identify, in the image, a reference object corresponding to the visual marker and, on the basis of the reference object, determine whether the surgical target has moved.

9. The surgical navigation system as claimed in claim 8, wherein the image comprises a first-time image generated by capturing the visual marker at a first point in time and a second-time image generated by capturing the visual marker at a second point in time later than the first point in time, and the controller is further configured to calculate first position information of the visual marker on the basis of the first-time image, calculate second position information of the visual marker on the basis of the second-time image, and determine whether the surgical target has moved on the basis of the first position information and the second position information.

10. The surgical navigation system as claimed in claim 8, wherein the controller is further configured to control movement of the robot arm on the basis of the reference object.

11. A surgical navigation system comprising:
a robot arm configured such that a surgical instrument is mounted at one end thereof;
a fixation pin configured to be fixed to a surgical target and comprising a visual marker;
a first camera configured to capture the visual marker from a first viewpoint and generate a first image;
a second camera configured to capture the visual marker from a second viewpoint different from the first viewpoint and generate a second image; and
a controller configured to identify, in the first image, a first reference object corresponding to the visual marker, identify, in the second image, a second reference object corresponding to the visual marker, control movement of the robot arm on the basis of the first reference object, and determine whether the surgical target has moved on the basis of the second reference object.

12. The surgical navigation system as claimed in claim 11, wherein the second image comprises:
a (2-1)-th image generated by capturing the visual marker at a first point in time; and
a (2-2)-th image generated by capturing the visual marker at a second point in time later than the first point in time, and
wherein the controller is further configured to calculate first position information of the visual marker on the basis of the (2-1)-th image, calculate second position information of the visual marker on the basis of the (2-2)-th image, and determine whether the surgical target has moved on the basis of the first position information and the second position information.

13. The surgical navigation system as claimed in claim 12, wherein, in response to determining that the surgical target has moved, the controller is further configured to output an alarm related to adjustment of a position of the surgical target or of the robot arm.

14. The surgical navigation system as claimed in claim 12, wherein the controller is further configured to calculate movement information of the visual marker on the basis of the first position information and the second position information and, in response to determining that the surgical target has moved, control movement of the robot arm on the basis of the movement information of the visual marker.

15. A method for controlling a surgical navigation system, the method being executed by at least one processor, comprising:
acquiring a medical image including at least a portion of a surgical target captured by a first imaging device;
generating a first image by capturing, at a first viewpoint via a first camera, a visual marker included in a fixation pin configured to be fixed to the surgical target;
generating a second image by capturing, at a second viewpoint different from the first viewpoint via a second camera, the visual marker;
identifying, in the first image, a first reference object corresponding to the visual marker;
identifying, in the second image, a second reference object corresponding to the visual marker;
controlling movement of a robot arm configured such that a surgical instrument is mounted at one end thereof on the basis of the first reference object; and
determining whether the surgical target has moved on the basis of the second reference object.
